(19) United States
(12) Patent Application Publication
(10) Pub. No.: US 2023/0414241 A1
(43) Pub. Date: Dec. 28, 2023

(54) TREATMENT SYSTEM AND METHOD OF OPERATING THE TREATMENT SYSTEM

(71) Applicant: OLYMPUS CORPORATION, Tokyo (JP)

(72) Inventors: Koichiro WATANABE, Higashiyamato-shi (JP); Kazuma TERAYAMA, Hanno-shi (JP); Tsuyoshi YAJI, Kawagoe-shi (JP); Misato KOBAYASHI, Tachikawa-shi (JP)

(73) Assignee: OLYMPUS CORPORATION, Tokyo (JP)

(21) Appl. No.: 18/243,137

(22) Filed: Sep. 7, 2023

Related U.S. Application Data
(63) Continuation of application No. PCT/JP2022/010123, filed on Mar. 8, 2022.
(60) Provisional application No. 63/159,108, filed on Mar. 10, 2021.

(51) Int. Cl. A61B 17/32; A61B 1/00

(52) U.S. Cl. CPC A61B 17/320068; A61B 1/000094; A61B 1/00045; A61B 2017/320069

(57) ABSTRACT

A treatment system includes: a treatment instrument configured to cut living tissue in a liquid; an endoscope configured to capture an endoscopic image including an image of the treatment instrument and an image of the living tissue; a support data storage configured to store, as support data to support a cutting treatment, at least one of data regarding a posture of the treatment instrument and image data on a location of a treatment section of the treatment instrument; a support data generator configured to generate support data to be displayed on a display based on the support data stored; a controller configured to control the display to display the endoscopic image; and a turbidity detector configured to detect a degree of cloudiness of the liquid.

### CROSS-REFERENCE TO RELATED APPLICATION

This application is a continuation of PCT International Application No. PCT/JP2022/010123 filed on Mar. 8, 2022 which is based on and claims priority under 35 U.S.C. § 119 to U.S. Provisional Application No. U.S. 63/159,108 filed on Mar. 10, 2021, the entire contents of which are incorporated herein by reference.

### BACKGROUND

### 1. Technical Field

The present disclosure relates to a treatment system and a method of operating the treatment system.

### 2. Related Art

Arthroscopic surgery is surgery in which a portal is formed in a joint to be treated, an arthroscope or a treatment instrument is inserted from the portal into the joint to be treated, and a treatment is performed while observing an inside of a joint cavity using the arthroscope in a situation where the joint cavity is filled with a perfusate. The arthroscopic surgery is performed using an arthroscopic surgery system (See, for example, WO 2018/078830 A1). WO 2018/078830 A1 also discloses an ultrasound treatment instrument for forming a hole in a bone. The ultrasound treatment instrument is configured such that a distal end of the treatment instrument ultrasonically vibrates. In the arthroscopic surgery, the distal end of the treatment instrument pulverizes (cuts) a bone by ultrasonic vibration, and a hole (bone hole) is formed in the bone. Thereafter, the two bone holes are connected to form one bone hole.

### SUMMARY

In some embodiments, a treatment system includes: a treatment instrument configured to cut living tissue in a liquid; an endoscope configured to capture an endoscopic image including an image of the treatment instrument and an image of the living tissue; a support data storage configured to store, as support data to support a cutting treatment, at least one of data regarding a posture of the treatment instrument and image data on a location of a treatment section of the treatment instrument; a support data generator configured to generate support data to be displayed on a display based on the support data stored; a controller configured to control the display to display the endoscopic image; and a turbidity detector configured to detect a degree of cloudiness of the liquid. The controller is configured to control the display to display the support data together with the endoscopic image, and perform display control to switch a display form of the support data generated by the support data generator in accordance with the degree of cloudiness of the liquid detected by the turbidity detector.

In some embodiments, provided is a method of operating a treatment system, the treatment system including a treatment instrument configured to cut living tissue in a liquid, an endoscope configured to capture an endoscopic image including an image of the treatment instrument and an image of the living tissue, a support data storage configured to store, as support data to support the cutting treatment, at least one of data regarding a posture of the treatment instrument and image data on a location of a treatment section of the treatment instrument, a support data generator configured to generate support data to be displayed on a display based on the support data stored, a controller configured to control the display to display the endoscopic image, and a turbidity detector configured to detect a degree of cloudiness of the liquid. The method includes: controlling, by the controller, the display to display the support data together with the endoscopic image; and performing, by the controller, display control to switch a display form of the support data generated by the support data generator in accordance with the degree of cloudiness of the liquid detected by the turbidity detector.

The above and other features, advantages and technical and industrial significance of this invention will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic configuration of a treatment system according to a first embodiment;

FIG. 2 is a diagram illustrating how to form a bone hole with an ultrasound probe;

FIG. 3A is a schematic view illustrating a schematic configuration of an ultrasound probe;

FIG. 3B is a schematic view in a direction of arrow A in FIG. 3A;

FIG. 3C is an enlarged view of a region R in FIG. 3A;

FIG. 4 is a block diagram illustrating an outline of a functional configuration of the treatment system according to the first embodiment;

FIG. 5 is a block diagram illustrating a functional configuration of an endoscope device;

FIG. 6A is a diagram schematically illustrating a state in which a field of view of an endoscope is good when a bone hole is formed in a lateral femoral condyle;

FIG. 6B is a diagram schematically illustrating a state in which a field of view of an endoscope is not good when a bone hole is formed in a lateral femoral condyle;

FIG. 7 is a block diagram illustrating a functional configuration of a treatment device;

FIG. 8 is a block diagram illustrating a functional configuration of a perfusion device;

FIG. 9 is a block diagram illustrating a functional configuration of an illumination device;

FIG. 10 is a flowchart illustrating an outline of treatment performed by a surgeon using the treatment system according to the first embodiment;

FIGS. 11A and 11B are diagrams for explaining a difference in appearance of a treatment instrument depending on whether a marker section is provided;

FIG. 12 is a diagram illustrating a configuration of an endoscope control device in a treatment system according to a second embodiment;

FIG. 13 is a flowchart illustrating an outline of cutting treatment in the treatment system according to the second embodiment;

FIGS. 14A to 14C are diagrams for explaining brightness at a distal end portion of a treatment instrument;

FIG. 15 is a flowchart illustrating an outline of cutting treatment in a treatment system according to a third embodiment;

FIG. 16 is a diagram for explaining an outline of cutting treatment in the treatment system according to the third embodiment;

FIG. 17 is a diagram (No. 1) illustrating an example of a display mode of a monitor in the treatment system according to the third embodiment;

FIG. 18 is a diagram (No. 2) illustrating an example of a display mode of a monitor in the treatment system according to the third embodiment; and

FIG. 19 is a diagram illustrating another example of a display mode of a monitor in the treatment system according to the third embodiment.

### DETAILED DESCRIPTION

Hereinafter, modes for carrying out the disclosure (hereinafter, referred to as embodiments) will be described with reference to the drawings. Note that the disclosure is not limited to the embodiments described below. Further, in the description of the drawings, the same parts are denoted by the same reference signs.

### First Embodiment

Schematic Configuration of Treatment System

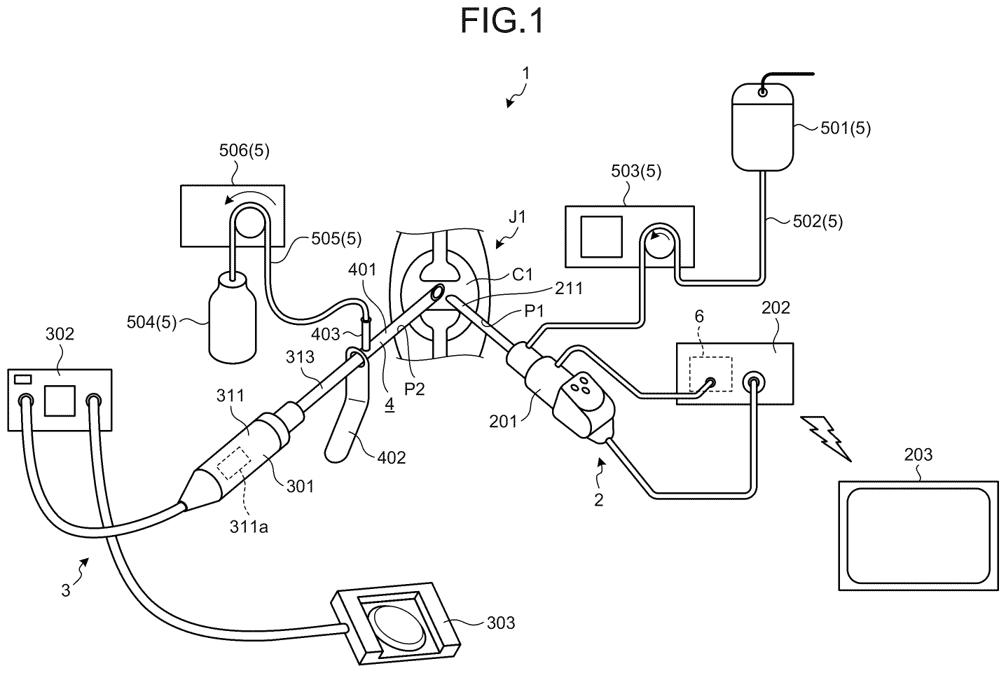

FIG. 1 is a diagram illustrating a schematic configuration of a treatment system 1 according to the first embodiment.

The treatment system 1 performs treatment on living tissue such as a bone by applying ultrasonic vibration to the living tissue. Here, the treatment means, for example, removal or cutting of living tissue such as a bone. Incidentally, FIG. 1 illustrates, as the treatment system 1, a treatment system that performs anterior cruciate ligament reconstructive surgery.

The treatment system 1 includes an endoscope device 2, a treatment device 3, a guiding device 4, a perfusion device 5, and an illumination device 6.

The endoscope device 2 includes an endoscope 201, an endoscope control device 202, and a display 203.

In the endoscope 201, a distal end portion of an insertion section 211 is inserted into a joint cavity C1 of a knee joint J1 through a first portal P1 through which an inside of the joint cavity C1 and an outside of skin communicate with each other. Then, the endoscope 201 captures illumination light that is applied to the inside of the joint cavity C1 and is reflected inside the joint cavity C1 (subject image), and captures the subject image.

The endoscope control device 202 performs various types of image processing on the captured image captured by the endoscope 201, and displays, on the display 203, the captured image that has been subjected to the image processing. The endoscope control device 202 is connected to the endoscope 201 and the display 203 in a wired or wireless manner.

The display 203 receives, via the endoscope control device 202, data, image data, audio data, and the like transmitted from each device of the treatment system, and displays/notifies the received data. The display 203 is configured using a liquid crystal display panel or an organic electro-luminescence (EL) display panel.

The treatment device 3 includes a treatment instrument 301, a treatment instrument control device 302, and a foot switch 303.

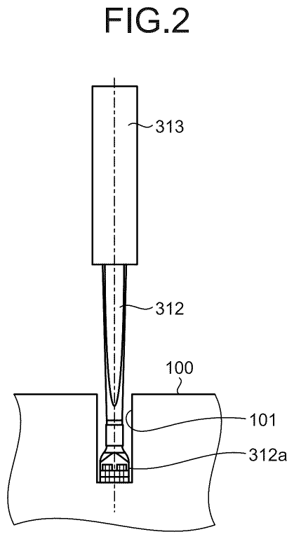

The treatment instrument 301 includes a treatment instrument body 311, an ultrasound probe 312 (see FIG. 2), and a sheath 313.

The treatment instrument body 311 is formed in a cylindrical shape. Then, an ultrasound transducer 311a (FIG. 1) is housed in the treatment instrument body 311. The ultrasound transducer 311a is configured by a bolt-clamped Langevin-type transducer and configured to generate ultrasonic vibration according to driving power supplied.

The treatment instrument control device 302 supplies the driving power to the ultrasound transducer 311a in accordance with operation on the foot switch 303 by a surgeon. Note that the supply of the driving power is not limited to the operation on the foot switch 303, and the supply of the drive power may be performed, for example, in accordance with operation on an operating unit (not illustrated) provided in the treatment instrument 301.

The foot switch 303 is an input interface for the surgeon to operate with his/her foot when the surgeon drives the ultrasound probe 312.

The guiding device 4, the perfusion device 5, and the illumination device 6 will be described later.

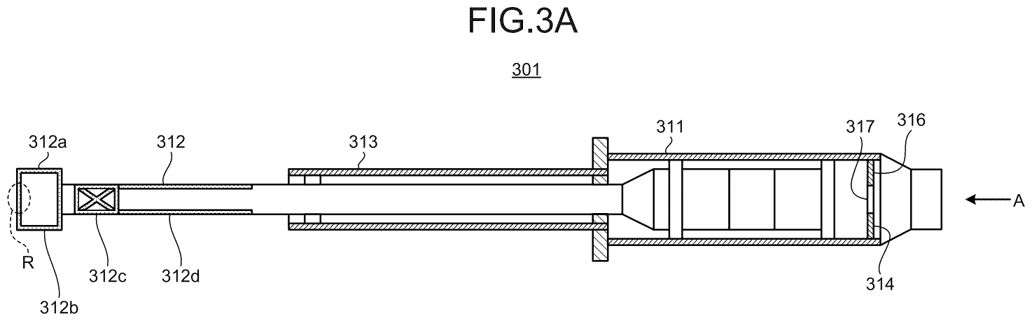

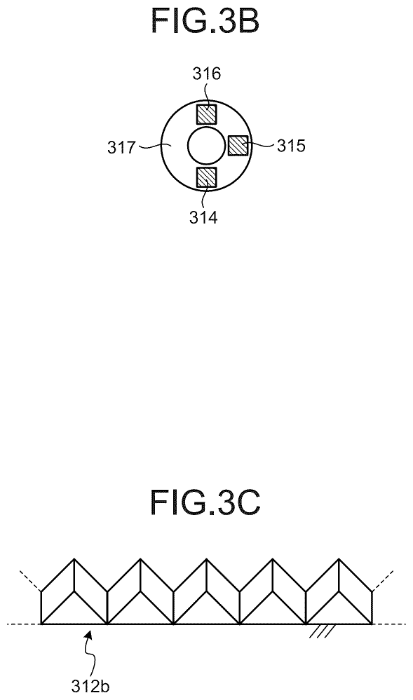

FIG. 2 is a diagram illustrating how to form a bone hole 101 with the ultrasound probe 312. FIG. 3A is a schematic view illustrating a schematic configuration of the ultrasound probe 312. FIG. 3B is a schematic view in a direction of arrow A in FIG. 3A. FIG. 3C is an enlarged view of a region R in FIG. 3A.

The ultrasound probe 312 is made of, for example, titanium alloy or the like, and has a substantially cylindrical shape. A proximal end portion of the ultrasound probe 312 is connected to the ultrasound transducer 311a in the treatment instrument body 311. Then, the ultrasound probe 312 transmits the ultrasonic vibration generated by the ultrasound transducer 311a from the proximal end to the distal end. In the first embodiment, the ultrasonic vibration is longitudinal vibration along the longitudinal direction (up-down direction in FIG. 2) of the ultrasound probe 312. As illustrated in FIG. 2, the ultrasound probe 312 has, at the distal end portion, a distal end treatment section 312a.

The sheath 313 is formed in a cylindrical shape longer than the treatment instrument body 311 and covers a part of the outer periphery of the ultrasound probe 312 from the treatment instrument body 311 to an arbitrary length.

The distal end portion of the ultrasound probe 312 in the treatment instrument 301 described above is inserted into the joint cavity C1 while being guided by the guiding device 4 inserted into the joint cavity C1 through a second portal P2 through which the inside of the joint cavity C1 and the outside of the skin communicate with each other.

Then, when ultrasonic vibration is generated in a state where the distal end treatment section 312a is in contact with a treatment target region 100 of the bone, a part of the bone mechanically colliding with the distal end treatment section 312a is pulverized into fine particles by the hammering action (see FIG. 2). Then, in response to the surgeon pushing the distal end treatment section 312a against the treatment target region 100, the distal end treatment section 312a moves forward into the treatment target region 100 while pulverizing the bone. As a result, the bone hole 101 is formed in the treatment target region 100.

In addition, the ultrasound probe 312 has, at the distal end portion, marker sections 312b to 312d (see FIG. 11B). Specifically, the marker section 312b is provided at a peripheral edge portion of the distal end treatment section 312a. The marker section 312c is provided on the proximal end side of the distal end treatment section 312a and includes a rectangular frame portion and an intersection portion formed in the frame portion and having an X-shape with intersecting diagonals. The marker section 312c is provided, for example, in a region where an aperture of the bone hole (aperture portion of the hole on the bone surface) can be positioned for a case where the ultrasound probe 312 is used to form the bone hole and the bone hole is completely formed. The marker section 312d extends in a longitudinal axis direction from the proximal end side of the marker section 312c. The marker sections 312b to 312d are subjected to processing of reflecting and scattering light, for example, retroreflection processing or knurling processing, or light emission processing such as fluorescent markers. For example, in a case where the retroreflection processing is performed on the marker section 312b, an uneven shape in which triangular prism-shaped spaces are continuously formed is formed (see FIG. 3C). Due to this uneven shape, light reflection is different from other portions, and reflected light returns to the light source (here, incidence of reflected light on the endoscope 201 is promoted), so that the visibility of the marker sections becomes higher than other portions.

At the proximal end of the treatment instrument body 311, an annular circuit board 317 on which a posture detection unit 314, a central processing unit (CPU) 315, and a memory 316 are mounted is provided (see FIGS. 3A and 3B). The posture detection unit 314 includes a sensor that detects rotation and movement of the treatment instrument 301. The posture detection unit 314 detects movement in three axial directions that are orthogonal to one another and include an axis parallel to the longitudinal axis of the ultrasound probe 312 and rotation around each axis. The posture detection unit 314 includes, for example, a triaxial angular velocity sensor (gyro sensor), an acceleration sensor, and the like. If the detection result of the posture detection unit 314 does not change for a certain period of time, then the treatment instrument control device 302 determines that the treatment instrument 301 is stationary. The CPU 315 corresponds to a control unit that controls the operation of the posture detection unit 314 and transmits and receives information to and from the treatment instrument control device 302.

In FIG. 1, the guiding device 4 is inserted into the joint cavity C1 through the second portal P2 and guides insertion of the distal end portion of the ultrasound probe 312 of the treatment instrument 301 into the joint cavity C1.

The guiding device 4 includes a guide body 401, a handle section 402, and a liquid discharge section 403 with a cock.

The guide body 401 has a tubular shape having a through hole through which the ultrasound probe 312 is inserted (see FIG. 1). The guide body 401 restricts the travel of the ultrasound probe 312 inserted into the through hole in a certain direction and guides the movement of the ultrasound probe 312. In the present embodiment, a cross-sectional shape orthogonal to the central axis on the outer peripheral surface and the inner peripheral surface of the guide body 401 is substantially circular.

The guide body 401 is tapered toward the distal end. That is, the distal end surface of the guide body 401 includes an aperture formed by a slope that obliquely intersects the central axis.

The liquid discharge section 403 with a cock is provided on the outer peripheral surface of the guide body 401 and has a cylindrical shape communicating with the inside of the guide body 401. One end of a liquid discharge tube 505 of the perfusion device 5 is connected to the liquid discharge section 403 with a cock, which forms a flow path that communicates the guide body 401 with the liquid discharge tube 505 of the perfusion device 5. The flow path is configured to open and close in response to an operation of a cock (not illustrated) provided in the liquid discharge section 403 with a cock.

The perfusion device 5 delivers a perfusate such as a sterilized physiological salt solution into the joint cavity C1 and discharges the perfusate to the outside of the joint cavity C1. The perfusion device 5 includes a liquid source 501, a liquid feed tube 502, a liquid feed pump 503, a liquid discharge bottle 504, a liquid discharge tube 505, and a liquid discharge pump 506 (see FIG. 1).

The liquid source 501 stores a perfusate.

The liquid feed tube 502 has one end connected to the liquid source 501 and the other end connected to the endoscope 201.

The liquid feed pump 503 feeds out the perfusate from the liquid source 501 toward the endoscope 201 through the liquid feed tube 502. Then, the perfusate fed out to the endoscope 201 is sent into the joint cavity C1 from a liquid feed hole formed at the distal end portion of the insertion section 211.

The liquid discharge bottle 504 stores the perfusate discharged to the outside of the joint cavity C1.

The liquid discharge tube 505 has one end connected to the guiding device 4 and the other end connected to the liquid discharge bottle 504.

The liquid discharge pump 506 follows the flow path of the liquid discharge tube 505 from the guiding device 4 inserted into the joint cavity C1, and discharges the perfusate in the joint cavity C1 to the liquid discharge bottle 504. In the first embodiment, the description will be given of the case using the liquid discharge pump 506, but the disclosure is not limited thereto, and a suction device provided in a facility may be used.

The illumination device 6 includes two light sources that respectively emit two illumination beams having different wavelength bands. The two illumination beams are, for example, white light and special light. The illumination beam from the illumination device 6 is propagated to the endoscope 201 via a light guide and emitted from the distal end of the endoscope 201.

Functional Configuration of Entire Treatment System

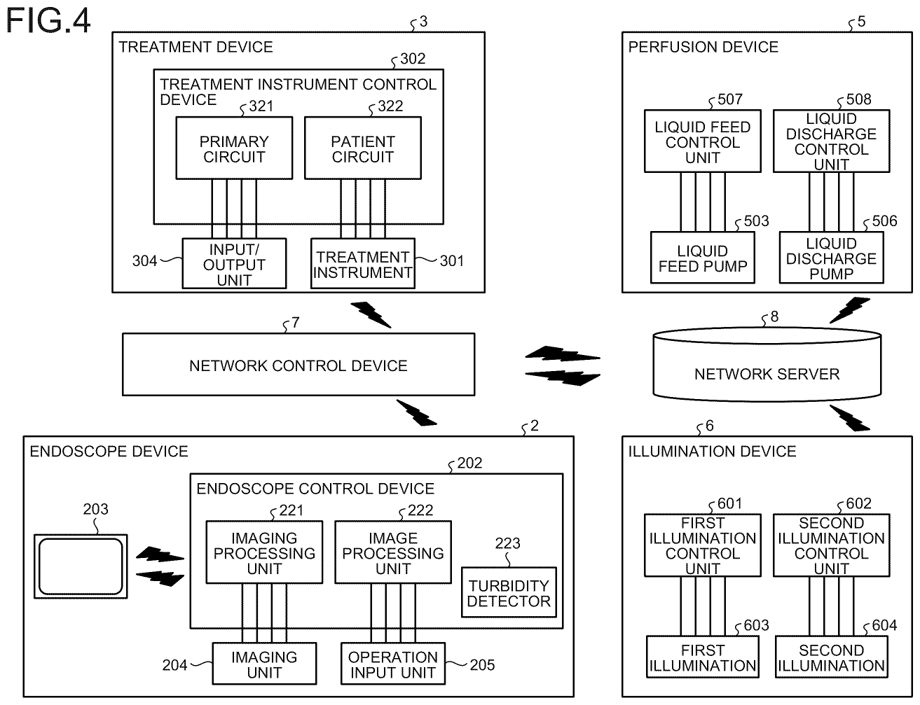

FIG. 4 is a block diagram illustrating an outline of a functional configuration of the entire treatment system. The treatment system 1 further includes a network control device 7 that controls communication of the entire system and a network server 8 that stores various data.

The network control device 7 is communicably connected to the endoscope device 2, the treatment device 3, the perfusion device 5, the illumination device 6, and the network server 8. Although FIG. 4 illustrates a case where the devices are wirelessly connected to each other, the devices may be connected to each other by wire. Hereinafter, the detailed functional configurations of the endoscope device 2, the treatment device 3, the perfusion device 5, and the illumination device 6 will be described.

Functional Configuration of Endoscope Device

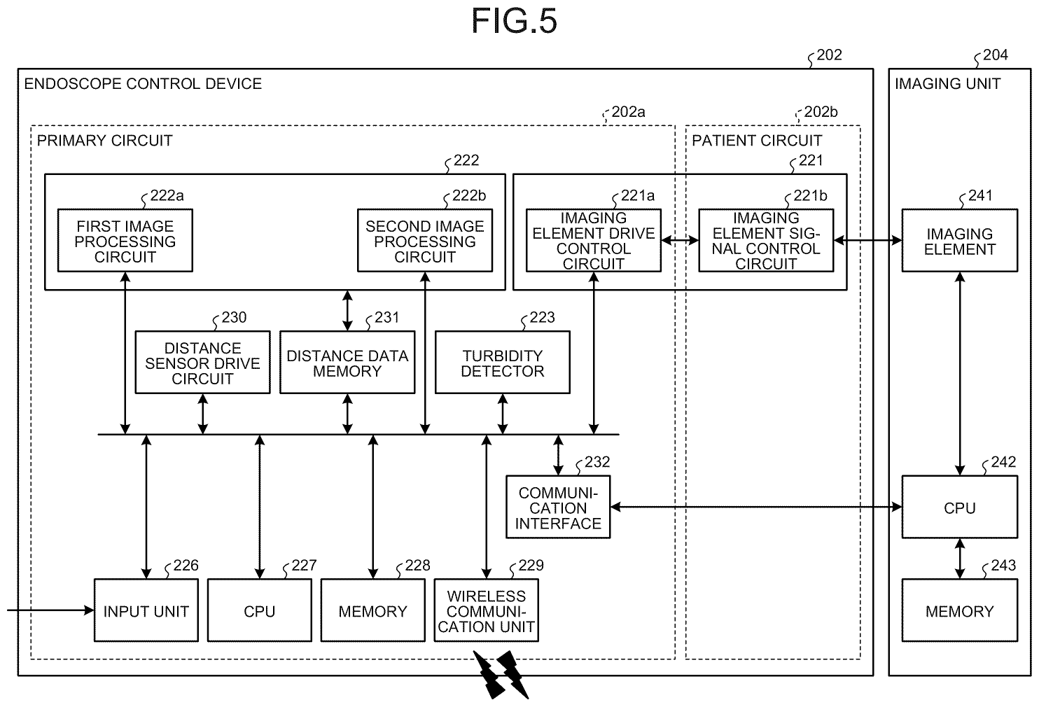

The endoscope device 2 includes the endoscope control device 202, the display 203, an imaging unit 204, and an operation input unit 205 (see FIGS. 4 and 5).

The endoscope control device 202 includes an imaging processing unit 221, an image processing unit 222, a turbidity detector 223, an input unit 226, a central processing unit (CPU) 227, a memory 228, a wireless communication unit 229, a distance sensor drive circuit 230, a distance data memory 231, and a communication interface 232.

The imaging processing unit 221 includes an imaging element drive control circuit 221a that drives and controls an imaging element 241 of the imaging unit 204, and an imaging element signal control circuit 221b that is provided in a patient circuit 202b electrically insulated from a primary circuit 202a to perform signal control of an imaging element 224a. The imaging element drive control circuit 221a is provided in the primary circuit 202a. Further, the imaging element signal control circuit 221b is provided in the patient circuit 202b electrically insulated from the primary circuit 202a.

The image processing unit 222 includes a first image processing circuit 222a that performs imaging processing and a second image processing circuit 222b that performs image editing processing.

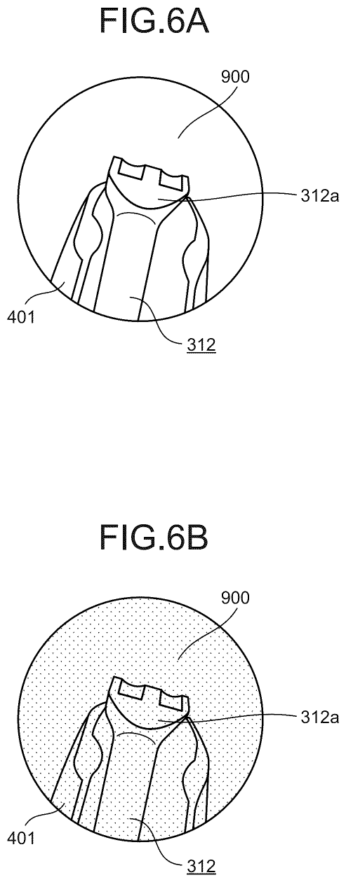

The turbidity detector 223 detects turbidity based on information regarding turbidity in the endoscope device 2. Here, the information regarding turbidity is, for example, a value obtained from imaging data generated by the endoscope 201, a physical property value of the perfusate, impedance or pH acquired from the treatment device 3, and the like. Here, FIGS. 6A and 6B are diagrams illustrating a state in which a field of view of the endoscope 201 is good and a state in which a field of view thereof is poor, respectively, and are diagrams schematically illustrating a field of view for a case where the surgeon forms a bone hole with respect to a lateral femoral condyle 900. Among these diagrams, FIG. 6B schematically illustrates a state in which the field of view is cloudy due to a bone pulverized into fine particles by the driving of the ultrasound probe 312. In FIG. 6B, fine bones are represented by dots. The fine bones are white, and the perfusate turns cloudy by particles of white particles containing the bones.

In FIG. 5, the input unit 226 receives an input of a signal input by the operation input unit 205.

The CPU 227 has control over the operation of the endoscope control device 202. The CPU 227 corresponds to a control unit that executes a program stored in the memory 228 to control the operation of each unit of the endoscope control device 202.

The memory 228 stores various types of information necessary for the operation of the endoscope control device 202, image data captured by the imaging unit 204, and the like.

The wireless communication unit 229 is an interface for performing wireless communication with other devices.

The distance sensor drive circuit 230 drives a distance sensor that measures a distance to a predetermined target object in an image captured by the imaging unit 204.

The distance data memory 231 stores distance data detected by the distance sensor.

The communication interface 232 is an interface for communicating with the imaging unit 204.

In the above-described configuration, components other than the imaging element signal control circuit 221b are provided in the primary circuit 202a, and are mutually connected by bus wiring.

The imaging unit 204 includes the imaging element 241, a CPU 242, and a memory 243.

The imaging element 241 is configured using a charge coupled device (CCD) or a complementary metal oxide semiconductor (CMOS).

The CPU 242 has control over the operation of the imaging unit 204. The CPU 242 corresponds to a control unit that executes a program stored in the memory 243 to control the operation of each unit of the imaging unit 204.

The memory 243 stores various types of information necessary for the operation of the imaging unit 204, image data, and the like.

In FIG. 4, the operation input unit 205 is configured using an input interface such as a mouse, a keyboard, a touch panel, or a microphone, and receives an operation input of the endoscope device 2 by the surgeon.

Functional Configuration of Treatment Device

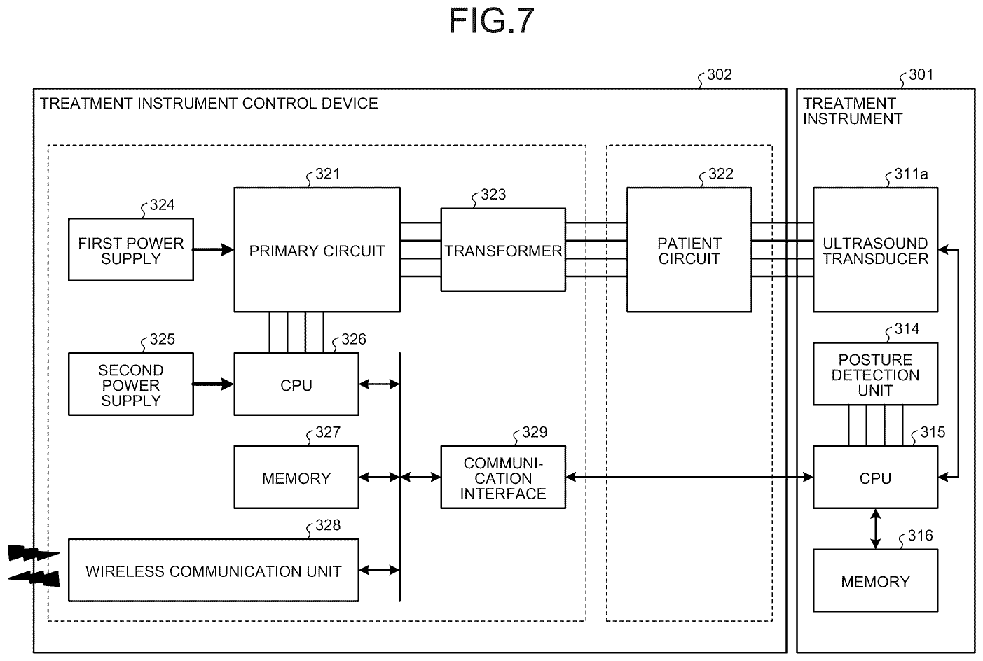

The treatment device 3 includes the treatment instrument 301, the treatment instrument control device 302, and an input/output unit 304 (see FIGS. 4 and 7).

The treatment instrument 301 includes the ultrasound transducer 311a, the posture detection unit 314, the CPU 315, and the memory 316 (see FIG. 7).

The posture detection unit 314 includes an acceleration sensor and/or an angular velocity sensor, and detects the posture of the treatment instrument 301.

The CPU 315 has control over the operation of the treatment instrument 301 including the ultrasound transducer 311a. The CPU 315 corresponds to a control unit that executes a program stored in the memory 316 to control the operation of each unit of the treatment instrument 301.

The memory 316 stores various types of information necessary for the operation of the treatment instrument 301.

The treatment instrument control device 302 includes a primary circuit 321, a patient circuit 322, a transformer 323, a first power supply 324, a second power supply 325, a CPU 326, a memory 327, a wireless communication unit 328, and a communication interface 329.

The primary circuit 321 generates power to be supplied to the treatment instrument 301.

The patient circuit 322 is electrically isolated from the primary circuit 321.

The transformer 323 electromagnetically connects the primary circuit 321 and the patient circuit 322.

The first power supply 324 is a high-voltage power supply that supplies driving power for the treatment instrument 301.

The second power supply 325 is a low-voltage power supply that supplies driving power for a control circuit in the treatment instrument control device 302.

The CPU 326 has control over the operation of the treatment instrument control device 302. The CPU 326 corresponds to a control unit that executes a program stored in the memory 327 to control the operation of each unit of the treatment instrument control device 302.

The memory 327 stores various types of information necessary for the operation of the treatment instrument control device 302.

The wireless communication unit 328 is an interface for performing wireless communication with other devices.

The communication interface 329 is an interface for communicating with the treatment instrument 301.

The input/output unit 304 is configured using an input interface such as a mouse, a keyboard, a touch panel, and a microphone and an output interface such as a monitor and a speaker, and outputs an operation input of the endoscope device 2 by the surgeon and various types of information to be notified to the surgeon (see FIG. 4).

Functional Configuration of Perfusion Device

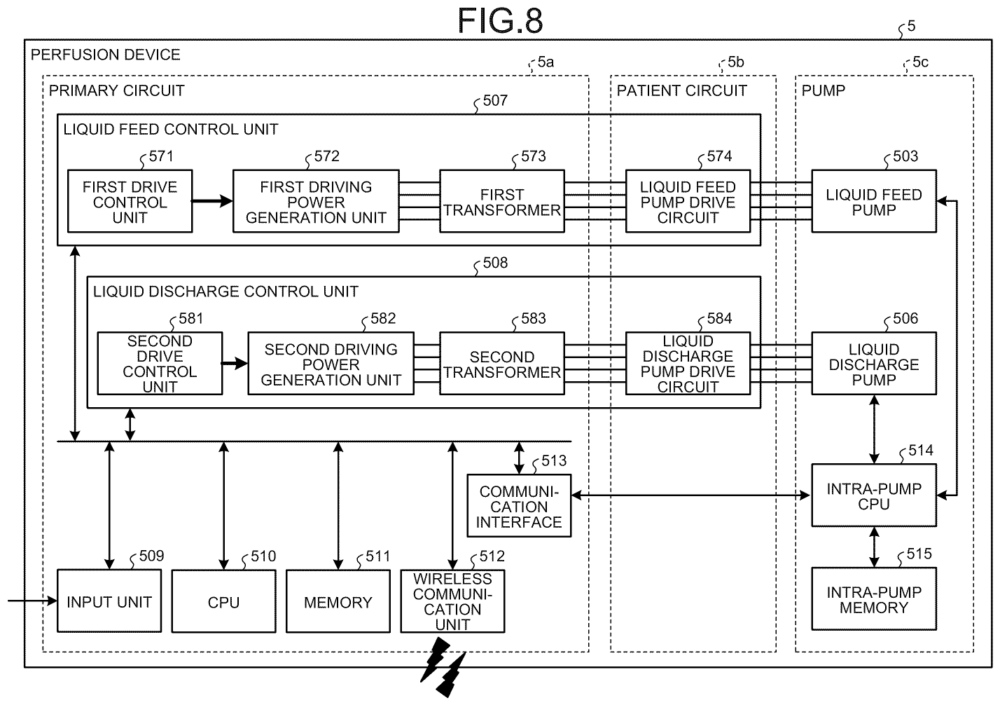

The perfusion device 5 includes the liquid feed pump 503, the liquid discharge pump 506, a liquid feed control unit 507, a liquid discharge control unit 508, an input unit 509, a CPU 510, a memory 511, a wireless communication unit 512, a communication interface 513, an intra-pump CPU 514, and an intra-pump memory 515 (see FIGS. 4 and 8).

The liquid feed control unit 507 includes a first drive control unit 571, a first driving power generation unit 572, a first transformer 573, and a liquid feed pump drive circuit 574 (see FIG. 8).

The first drive control unit 571 controls the drive for the first driving power generation unit 572 and the liquid feed pump drive circuit 574.

The first driving power generation unit 572 generates driving power for the liquid feed pump 503.

The first transformer 573 electromagnetically connects the first driving power generation unit 572 and the liquid feed pump drive circuit 574.

The first drive control unit 571, the first driving power generation unit 572, and the first transformer 573 are provided in a primary circuit 5a. Further, the liquid feed pump drive circuit 574 is provided in a patient circuit 5b electrically insulated from the primary circuit 5a.

The liquid discharge control unit 508 includes a second drive control unit 581, a second driving power generation unit 582, a second transformer 583, and a liquid discharge pump drive circuit 584.

The second drive control unit 581 controls the drive for the second driving power generation unit 582 and the liquid discharge pump drive circuit 584.

The second driving power generation unit 582 generates driving power for the liquid discharge pump 506.

The second transformer 583 electromagnetically connects the second driving power generation unit 582 and the liquid discharge pump drive circuit 584.

The second drive control unit 581, the second driving power generation unit 582, and the second transformer 583 are provided in the primary circuit 5a. Further, the liquid discharge pump drive circuit 584 is provided in the patient circuit 5b.

The input unit 509 receives inputs of various signals such as an operation input (not illustrated).

The CPU 510 and the intra-pump CPU 514 cooperate to control over the operation of the perfusion device 5. The CPU 510 corresponds to a control unit that executes a program stored in the memory 511 to control the operation of each unit of the perfusion device 5 via a BUS line.

The memory 511 stores various types of information necessary for the operation of the perfusion device 5.

The wireless communication unit 512 is an interface for performing wireless communication with other devices.

The communication interface 513 is an interface for communicating with the intra-pump CPU 514.

The intra-pump memory 515 stores various types of information necessary for the operation of the liquid feed pump 503 and the liquid discharge pump 506.

The input unit 509, the CPU 510, the memory 511, the wireless communication unit 512, and the communication interface 513 are provided in the primary circuit 5a.

The intra-pump CPU 514 and the intra-pump memory 515 are provided in a pump 5c. The intra-pump CPU 514 and the intra-pump memory 515 may be provided around the liquid feed pump 503 or may be provided around the liquid discharge pump 506.

Functional Configuration of Illumination Device

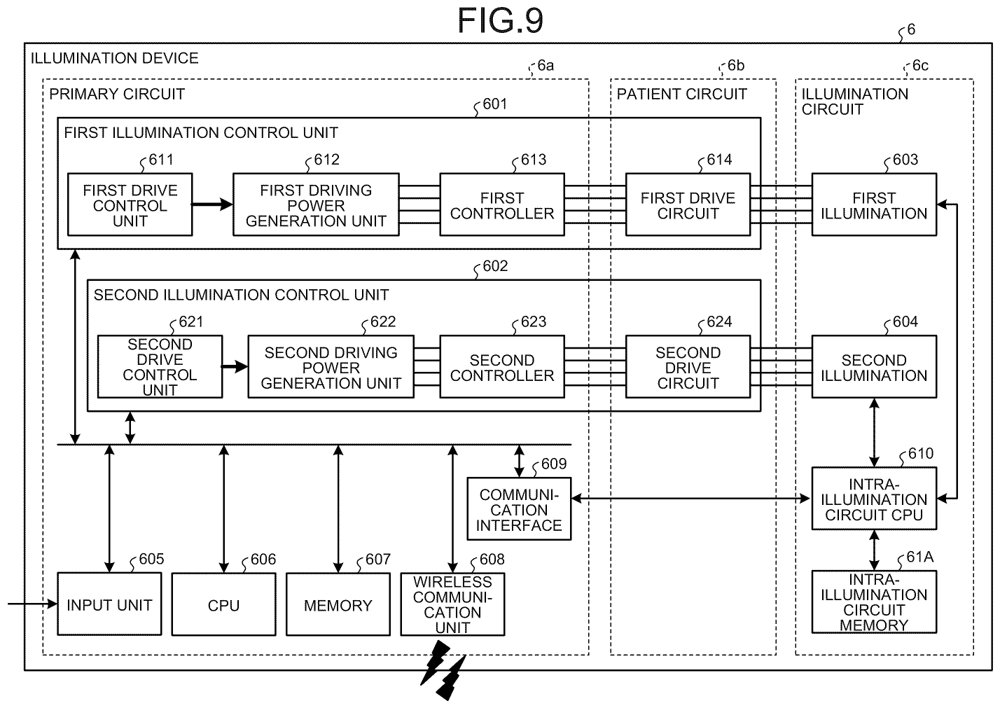

The illumination device 6 includes a first illumination control unit 601, a second illumination control unit 602, first illumination 603, second illumination 604, an input unit 605, a CPU 606, a memory 607, a wireless communication unit 608, a communication interface 609, an intra-illumination circuit CPU 610, and an intra-illumination circuit memory 61A (see FIGS. 4 and 9).

The first illumination control unit 601 includes a first drive control unit 611, a first driving power generation unit 612, a first controller 613, and a first drive circuit 614.

The first drive control unit 611 controls the drive for the first driving power generation unit 612, the first controller 613, and the first drive circuit 614.

The first driving power generation unit 612 generates driving power for the first illumination 603.

The first controller 613 controls the light output of the first illumination 603.

The first drive circuit 614 drives the first illumination 603 to output illumination light.

The first drive control unit 611, the first driving power generation unit 612, and the first controller 613 are provided in a primary circuit 6a. Further, the first drive circuit 614 is provided in a patient circuit 6b electrically insulated from the primary circuit 6a.

The second illumination control unit 602 includes a second drive control unit 621, a second driving power generation unit 622, a second controller 623, and a second drive circuit 624.

The second drive control unit 621 controls the drive for the second driving power generation unit 622, the second controller 623, and the second drive circuit 624.

The second driving power generation unit 622 generates driving power for the second illumination 604.

The second controller 623 controls the light output of the second illumination 604.

The second drive circuit 624 drives the second illumination 604 to output illumination light.

The second drive control unit 621, the second driving power generation unit 622, and the second controller 623 are provided in the primary circuit 6a. The second drive circuit 624 is provided in the patient circuit 6b.

The input unit 605 receives inputs of various signals such as an operation input (not illustrated).

The CPU 606 and the intra-illumination circuit CPU 610 cooperate to control over the operation of the illumination device 6. The CPU 606 corresponds to a control unit that executes a program stored in the memory 607 to control the operation of each unit of the illumination device 6.

The memory 607 stores various types of information necessary for the operation of the illumination device 6.

The wireless communication unit 608 is an interface for performing wireless communication with other devices.

The communication interface 609 is an interface for communicating with an illumination circuit 6c.

The intra-illumination circuit memory 61A stores various types of information necessary for the operation of the first illumination 603 and the second illumination 604.

The input unit 605, the CPU 606, the memory 607, the wireless communication unit 608, and the communication interface 609 are provided in the primary circuit 6a.

The intra-illumination circuit CPU 610 and the intra-illumination circuit memory 61A are provided in the illumination circuit 6c.

Outline of Treatment

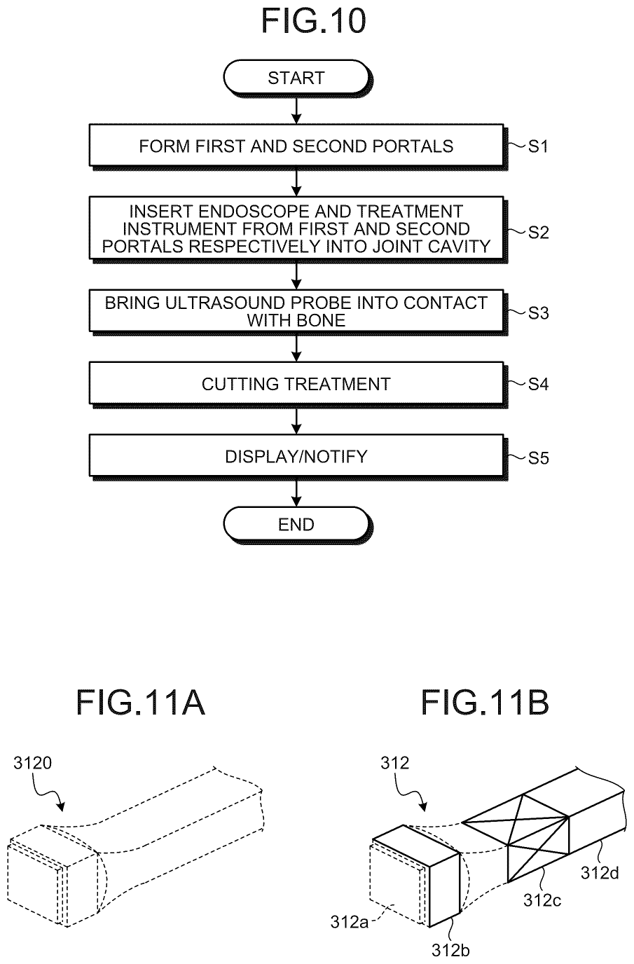

FIG. 10 is a flowchart illustrating the outline of treatment performed by a surgeon using the treatment system 1. The surgeon who performs the treatment may be one doctor or two or more doctors and assistants.

First, the surgeon forms a first portal P1 and a second portal P2 each of which communicates the inside of the joint cavity C1 of the knee joint J1 with the outside of the skin (Step S1).

Subsequently, the surgeon inserts the endoscope 201 into the joint cavity C1 from the first portal P1, inserts the guiding device 4 into the joint cavity C1 from the second portal P2, and inserts the treatment instrument 301 into the joint cavity C1 in accordance with a guide by the guiding device 4 (Step S2). Here, the case where the two portals are formed first, and then the endoscope 201 and the treatment instrument 301 are inserted into the joint cavity C1 from each of the portals has been described. However, it is also possible that the first portal P1 is formed and the endoscope 201 is first inserted into the joint cavity C1, and then the second portal P2 is formed and the guiding device 4 and the treatment instrument 301 are inserted into the joint cavity C1.

Thereafter, the surgeon brings the ultrasound probe 312 into contact with a bone to be treated while visually checking an endoscopic image for the inside of the joint cavity C1 displayed on the display 203 (Step S3).

Subsequently, the surgeon performs cutting treatment using the treatment instrument 301 (Step S4). At this time, light is reflected from the marker sections 312b to 312d by the illumination from the illumination device 6. This reflection facilitates visual recognition of the marker sections 312b to 312d.

FIGS. 11A and 11B are diagrams for explaining a difference in appearance of a treatment instrument depending on whether a marker section is provided. As illustrated in FIG. 11A, in a conventional ultrasound probe 3120 having no marker sections 312b to 312d, it is difficult to visually recognize the ultrasound probe 3120 due to turbidity. In contrast, as illustrated in FIG. 11B, in the ultrasound probe 312 including the marker sections 312b to 312d, it is easy to visually recognize the marker sections even when turbidity occurs because the marker sections reflect or scatter the illumination light, for example.

At this time, the image processing unit 222 that generates an endoscopic image corresponds to a support data generator that generates, as support data, display data regarding an image in the location of the treatment section. Further, the endoscopic image generated by the image processing unit 222 is stored in the memory 228 serving as a support data storage.

Thereafter, the display 203 displays the inside of the joint cavity C1 and performs processing of displaying/notifying information regarding the state after the cutting treatment (Step S5). For example, the endoscope control device 202 stops the display/notification after a predetermined time after the display/notification processing.

In the first embodiment described above, the configuration is provided in which the ultrasound probe 312 includes the marker sections 312b to 312d to ensure the visibility of the marker sections even during treatment. A user of the treatment instrument 310 visually recognizes the marker sections to thereby be able to grasp the position of the ultrasound probe 312 and the depth at which the ultrasound probe 312 penetrates into the bone even in a cloudy state due to bone powder. According to the first embodiment, the visibility of the treatment instrument 301 in a white turbid liquid is improved, which makes it possible to suppress the influence on the surgery caused by the turbidity in the perfusate.

### Second Embodiment

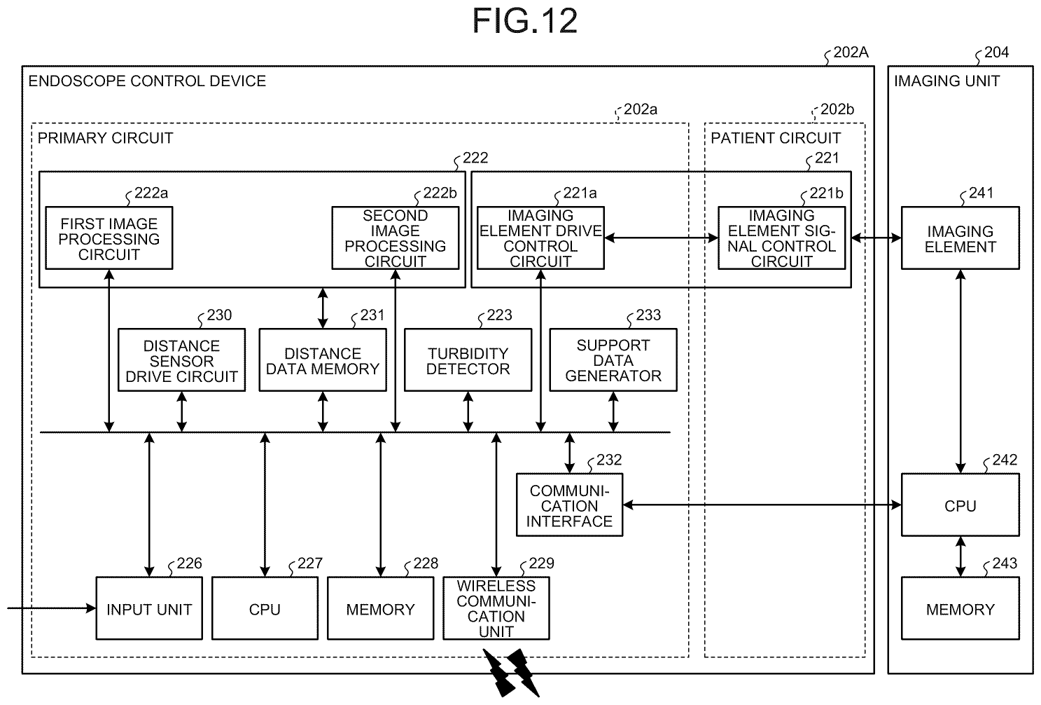

The description goes on to the second embodiment with reference to FIGS. 12 to 14C. In the first embodiment, the example in which the user visually recognizes the ultrasound probe 312 by light scattering or light emission of the marker sections of the treatment instrument 301 has been described, but in the second embodiment, an example in which processing of enhancing the marker sections is performed on an endoscopic image acquired by the endoscope 201 will be described.

FIG. 12 is a diagram illustrating a configuration of an endoscope control device in a treatment system according to the second embodiment. An endoscope control device 202A according to the second embodiment further includes a support data generator 233 in addition to the endoscope control device 202 according to the first embodiment. Since the configuration other than the support data generator 233 is similar to the configuration of the treatment system 1, the description thereof will be omitted.

The support data generator 233 generates, as support data, an image that is to be displayed on the display 203 and is to support the treatment performed by the user of the treatment instrument 301. In the second embodiment, an example will be described in which the support data generator 233 generates, as the support data, an enhanced image in which a part (here, a marker section) of the treatment instrument 301 is enhanced.

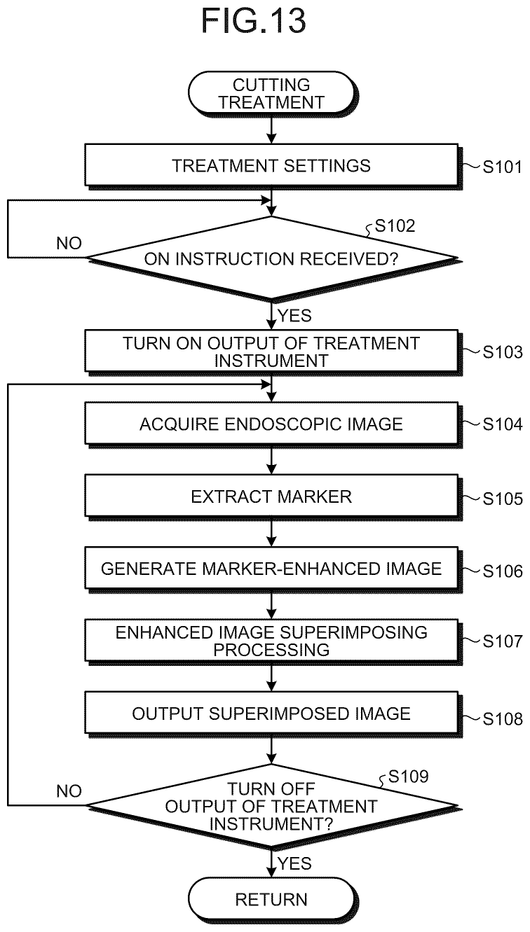

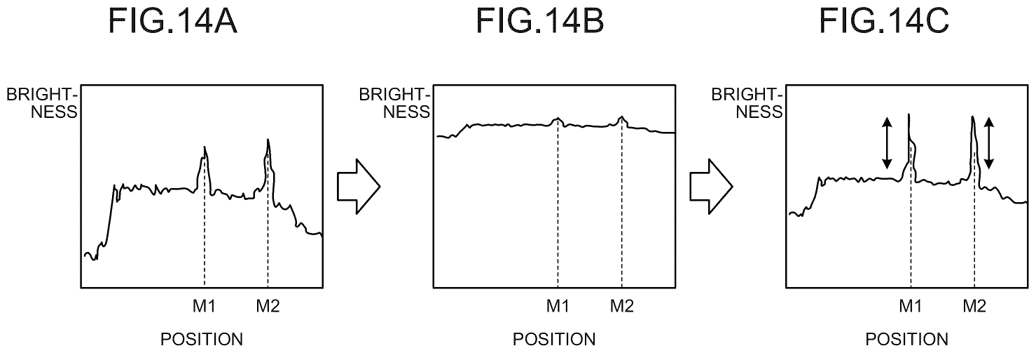

In the second embodiment, the treatment is performed in the flow illustrated in FIG. 10. Hereinafter, cutting treatment according to the second embodiment will be described. FIG. 13 is a flowchart illustrating an outline of the cutting treatment in the treatment system according to the second embodiment. FIGS. 14A to 14C are diagrams for explaining brightness at a distal end portion of the treatment instrument. The following describes a case where the CPU of each control device performs communication with one another and perform cooperative control to execute each processing, but for example, any of the control devices such as the network control device 7 may collectively execute the processing.

The CPU 326 of the treatment instrument control device 302 performs treatment settings such as a mode of cutting to be executed by the treatment instrument 301 (Step S101). In setting the cutting mode, for example, a frequency of ultrasonic vibration or the like is set.

The CPU 326 determines whether an input of an ON instruction for the treatment instrument 301 has been received (Step S102). For example, the CPU 326 determines whether a signal is input from the foot switch 303. If determining that the input of the ON instruction for the treatment instrument 301 has not been received (Step S102: No), then the CPU 326 repeats to confirm the input of the ON instruction. On the other hand, if the CPU 326 determines that the input of the ON instruction for the treatment instrument 301 has been received (Step S102: Yes), then the processing proceeds to Step S103.

In Step S103, the CPU 326 turns on the output of the treatment instrument 301 and vibrates the ultrasound probe 312.

Thereafter, the CPU 227 of the endoscope control device 202 performs control to acquire the endoscopic image captured by the imaging unit 204 (Step S104).

After acquiring the endoscopic image, the CPU 227 instructs the support data generator 233 to perform marker extraction (Step S105). After extracting the marker, the support data generator 233 generates a marker-enhanced image in which the marker sections are enhanced (Step S106) The support data generator 233 executes, for example, gradation correction processing for correcting gradation of a part corresponding to the image of the treatment instrument 301. In the second embodiment, the brightness difference is increased by increasing the brightness expression width. The generated marker-enhanced image is temporarily stored in the memory 228 as support data for supporting cutting. That is, the memory 228 constitutes the support data storage. Then, the second image processing circuit 222b reads the enhanced image from the memory 228, and generates, as the display data, a superimposed image obtained by superimposing the marker-enhanced image on the corresponding endoscopic image (Step S107).

FIGS. 14A to 14C are diagrams for explaining brightness at a position of the distal end portion of the treatment instrument 301 based on data on captured image. FIG. 14A illustrates the brightness of the distal end portion of the ultrasound probe 312 whose image is captured in a state before turning cloudy due to bone powder before treatment. FIG. 14B illustrates the brightness of the distal end portion of the ultrasound probe 312 whose image is captured in a cloudy state due to treatment. FIG. 14C illustrates the brightness of the distal end portion of the ultrasound probe 312 for a case where the gradation correction is performed on the brightness of the image in FIG. 14B. In the endoscopic image before cloudiness, since the brightness of the marker sections located at positions M1 and M2 is brighter than that at other portions, the ultrasound probe 312 can be easily visually recognized (see FIG. 14A). On the other hand, in the cloudy state, the image is bright as a whole, and the difference in brightness decreases (see FIG. 14B). In this image, the visibility of the ultrasound probe 312 decreases, and the processing is continued after the cloudiness subsides. To cope with this, the gradation correction is performed on a region including the positions M1 and M2 to increase the difference in brightness (for example, the arrow portions in the drawing), and the visibility of the marker sections is improved (see FIG. 14C).

Returning to FIG. 13, after the support data generator 233 generates the superimposed image, the CPU 227 causes the display 203 to display the superimposed image (Step S108). On the display 203, an image in which the marker sections 312b to 312d are enhanced more than in a normal image is displayed.

Thereafter, the CPU 326 of the treatment instrument control device 302 determines whether the output of the treatment instrument 301 is turned off (Step S109). If the CPU 326 does not determine that the output of the treatment instrument 301 is turned off (Step S109: No), then the processing proceeds to Step S104 and the CPU 227 is instructed via communication to execute, as for a new endoscopic image, processing for creating a superimposed image and displaying the same. During the cutting treatment, the processing for displaying the superimposed image is repeatedly executed at predetermined time intervals or continuously. On the other hand, if the CPU 326 determines that the output of the treatment instrument 301 is turned off (Step S109: Yes), then the processing returns to Step S5 illustrated in FIG. 10.

In the second embodiment described above, as with the first embodiment, the configuration is provided in which the ultrasound probe 312 includes the marker sections 312b to 312d to ensure the visibility of the marker sections even during treatment. A user of the treatment instrument 310 visually recognizes the marker sections to thereby be able to grasp the position of the ultrasound probe 312 and the depth at which the ultrasound probe 312 penetrates into the bone even in a cloudy state due to bone powder. According to the second embodiment, it is possible to reduce the influence on surgery due to turbidity in a perfusate.

Further, in the second embodiment, the marker sections are extracted based on the brightness of the image, and the image in which the marker sections are enhanced is displayed, so that the visibility of the marker sections can be further enhanced.

### Third Embodiment

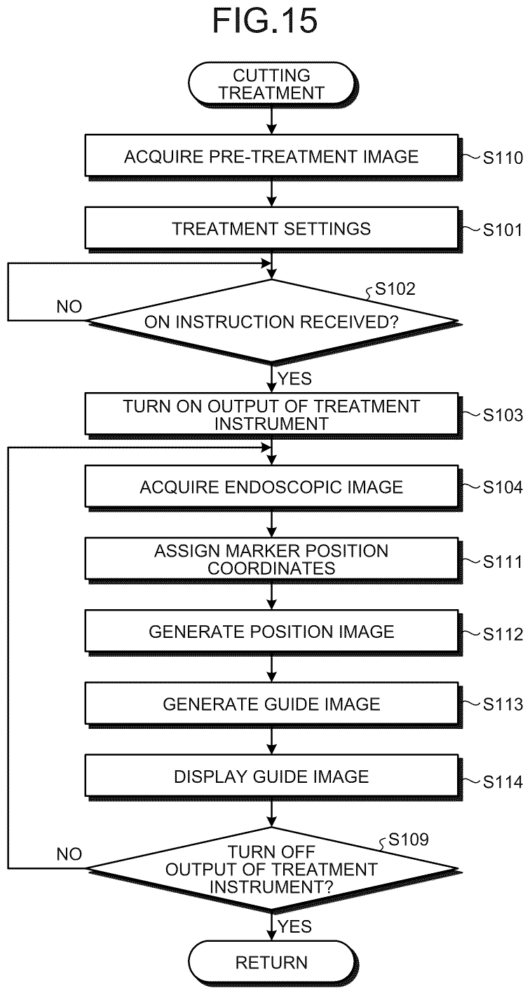

The description goes on to the third embodiment with reference to FIGS. 15 to 19. In the first embodiment, the example in which the markers are used to visually recognize the position of the ultrasound probe 312 as the cutting preparation detection for the treatment instrument 301 has been described. However, in the third embodiment, an example in which the spatial position of the ultrasound probe 312 is displayed will be described. Since the configuration of the treatment system is similar to that of the second embodiment, the description thereof will be omitted.

In the third embodiment, an example in which a position image indicating a relative positional relationship between a treatment target position and a position of the treatment instrument 301 (here, a position of the distal end treatment section 312a) is generated as the support data will be described.

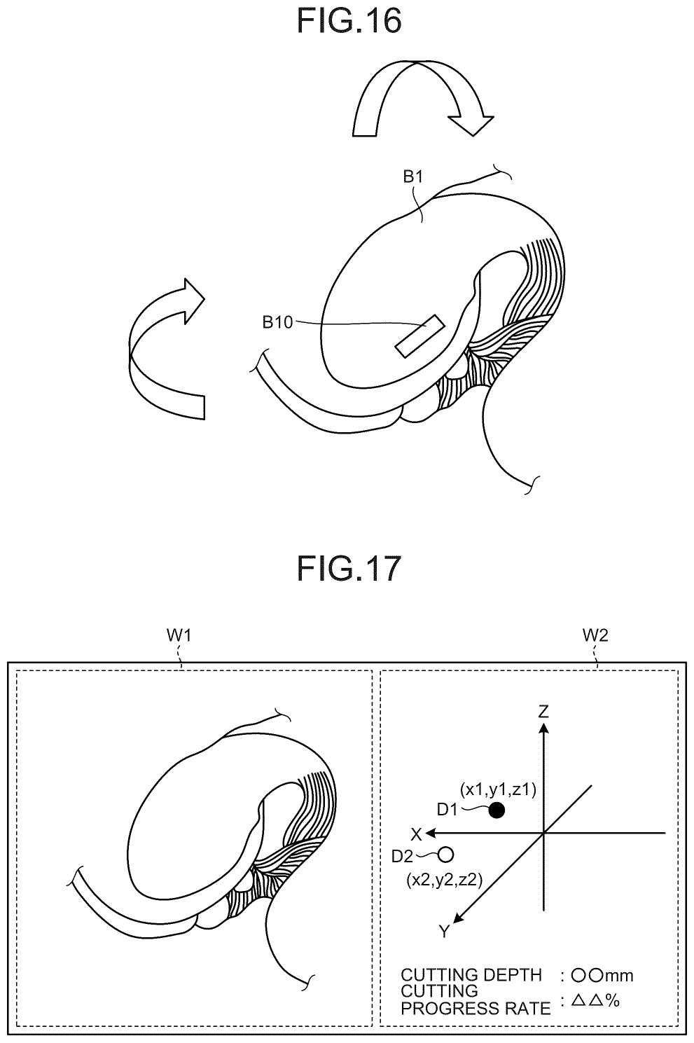

In the third embodiment, the processing is executed according to the flowcharts illustrated in FIGS. 10 and 12. Hereinafter, processing different from that in the flowcharts illustrated in FIGS. 10 and 13 will be described. FIG. 15 is a flowchart illustrating an outline of the cutting treatment in the treatment system according to the third embodiment. FIG. 16 is a diagram for explaining an outline of the cutting treatment in the treatment system according to the third embodiment. In the treatment, the user sets a cutting depth in advance.

In the third embodiment, as a pre-treatment image, the entire circumferential image of a treatment site is acquired in advance before treatment (Step S110). The entire circumferential image is acquired by, for example, the endoscope 201. Specifically, in a case where the endoscope 201 is an oblique endoscope, the entire circumferential image is acquired by capturing an image of the treatment site around two axes orthogonal to each other (see the arrows in FIG. 16). In a case where the endoscope 201 includes a fisheye lens, the entire circumferential image can be acquired only by imaging in one direction.

Note that space coordinates correlated with a space including the treatment site may be assigned to the entire circumferential image. In addition, for example, the position of the treatment target may be registered to the entire circumferential image. The generated entire circumferential image is temporarily stored in the memory 228 as support data for supporting cutting. That is, the memory 228 constitutes the support data storage. The support data generator 233 generates display data for performing support display based on the support data temporarily stored in the memory 228. A region B10 illustrated in FIG. 16 indicates a region (treatment target position) where a bone hole is formed.

In Steps S105 to S108 of FIG. 13, instead of the enhanced image generation processing, processing for generating the support data and a guide image is executed. Here, in the third embodiment, three-dimensional space coordinates are assigned to the treatment instrument 301 (representative position of the marker sections) (Step S111). The support data generator 233, for example, plots the position coordinates of the treatment instrument 301 in the space coordinates.

Then, the support data generator 233 executes processing for creating a position image indicating a relative position between the treatment target position and the representative position of the marker sections (Step S112). At this time, the support data generator 233 generates a position image in which the representative position of the marker sections and the treatment target position corresponding to the set cutting depth are plotted in the coordinate space based on the coordinates of the representative position of the marker sections. As the treatment target position, a position (coordinates) is set which is away from the position of the treatment instrument 301 (representative position of the marker sections) by the preset cutting depth. Further, in order to easily grasp the cutting situation, the support data generator 233 generates data indicating the cutting depth and a cutting progress rate to a cutting completion position. Note that display/non-display can be set for the cutting depth and the cutting progress rate.

As the position of the treatment instrument 301, coordinates of the position of the treatment instrument 301 and the treatment target position in the coordinate space are set based on posture data and a movement direction detected by the posture detection unit 314 until the treatment instrument 301 is brought into a stationary state immediately before being driven. In the above description, the detected posture data, movement direction, and so on are temporarily stored in the memory 228 as the support data for supporting cutting. That is, the memory 228 constitutes the support data storage.

Here, the display direction of the coordinates on an endoscopic image display area W1 and a position image display area W2 may be fixed at the reference direction, or may be adjustable so as to be changeable to an arbitrary direction that a surgeon can intuitively grasp easily (see FIG. 17).

A distance measured by the distance sensor drive circuit 230 may be used as necessary.

Thereafter, the second image processing circuit 222b generates a guide image to be displayed on the display 203 (Step S113). The guide image includes an endoscopic image and a position image. Then, the CPU 227 outputs the guide image and displays the generated guide image on the display 203 (Step S114).

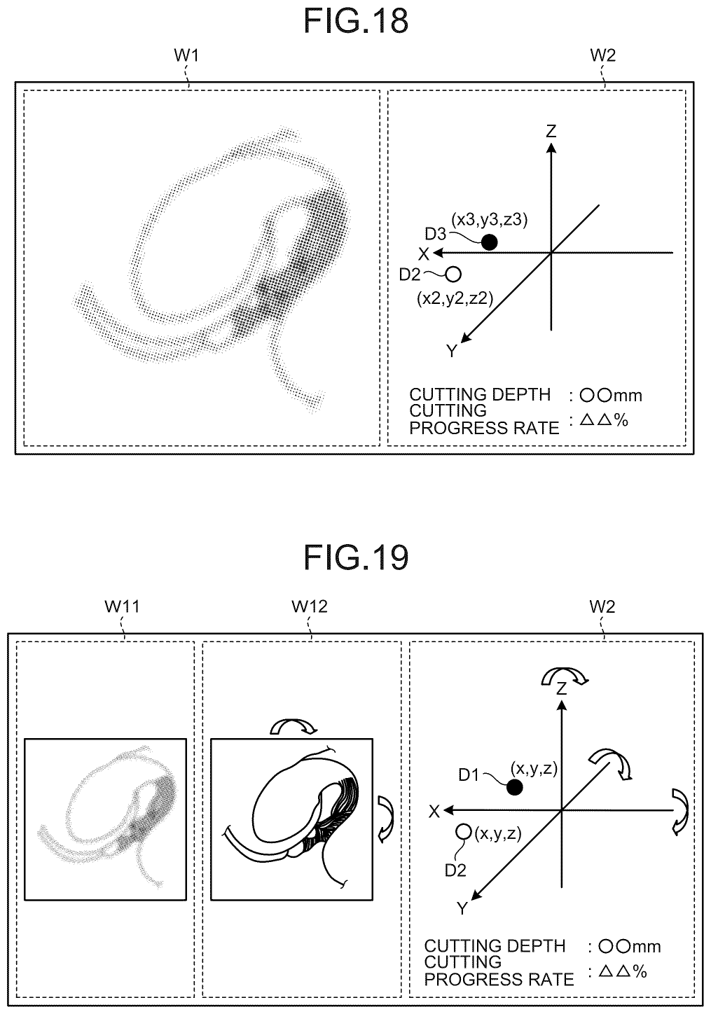

FIGS. 17 and 18 are diagrams illustrating an example of a display mode of a monitor in the treatment system according to the third embodiment. On the display screen of the display 203, for example, a guide image is displayed (see FIG. 17). The guide image includes the endoscopic image display area W1 in which an endoscopic image is displayed, and the position image display area W2 in which a relative positional relationship between the position of the treatment target and the position of the marker section is indicated. In the position image, a position D1 (x1, y1, z1) of the marker section and a treatment target position D2 (x2, y2, z2) are displayed on the space coordinates. Even in a case where the endoscopic image turns cloudy due to the treatment (see FIG. 18), the ultrasound probe 312 can be operated to the treatment target position by confirming the position D3 (x3, y3, z3) of the marker section. At this time, a rough distance can be grasped based on the displayed coordinates. In addition, a distance between the coordinates may be calculated, and the distance or a distance converted into an actual distance may be displayed.

In the position image display area W2, the coordinate axes may be hidden. In addition, the coordinate system may be rotated in a direction in which the user can intuitively grasp when viewing the display screen. In this case, for example, the direction of a line-of-sight of the user with respect to the display screen may be set in advance, and the coordinate system may be rotated in a direction in which the marker section and the treatment position are aligned in the line-of-sight direction. Alternatively, a line-of-sight detector may be actually provided on the display screen to actually detect a line-of-sight of the user, and the coordinate system may be rotated in a direction in which the marker section and the treatment position are aligned in the direction of the detected line-of-sight.

In the third embodiment described above, as with the first embodiment, the configuration is provided in which the ultrasound probe 312 includes the marker sections 312b to 312d to ensure the visibility of the marker sections even during treatment. The user of the treatment instrument 310 uses an image to visually recognize the relative position of the marker sections to thereby be able to grasp the position of the ultrasound probe 312 and the depth at which the ultrasound probe 312 penetrates into the bone even in a cloudy state due to bone powder. According to the third embodiment, the state immediately before the treatment is detected and controlled, which makes it possible to suppress the influence on the surgery caused by turbidity in the perfusate.

Further, in the third embodiment, since the relative position to the target position is displayed together with the endoscopic image, the user can operate the ultrasound probe 312 with respect to the target position even in a case where the visibility of the treatment instrument 301 in the endoscopic image is reduced.

Here, the display mode of the guide image is not limited to the images illustrated in FIGS. 17 and 18. For example, the endoscopic image at the time of treatment and the image before the treatment may be displayed side by side. Alternatively, in order to indicate the degree of allowable positional deviation with respect to the current position and the treatment completion position of the treatment instrument 301, data indicating an allowable movement range may be generated and displayed in a superimposed manner. FIG. 19 is a diagram illustrating another example of a display mode of a monitor in the treatment system according to the third embodiment. On the display screen illustrated in FIG. 19, for example, a guide image is displayed. The guide image includes an endoscopic image display area W1l in which an endoscopic image is displayed, a pre-treatment image display area W12 in which an image of a treatment site before treatment is displayed, and the position image display area W2 in which a relative positional relationship between the position of the treatment target and the position of the marker section is indicated. The image of the treatment site displayed in the pre-treatment image display area W12 is an entire circumferential image, and the image can be rotated by inputting an instruction signal via the input/output unit 304. Further, the space coordinates of the position image also rotate in conjunction with the rotation of the image of the treatment site. According to this rotation, the position D1 of the marker section and the treatment target position D2 also move.

The pre-treatment image display area W12 may be always displayed, or may be displayed only when a display instruction is input.

In the third embodiment, the example of detecting the position of the treatment instrument 301 using retroreflected light from the marker sections has been described. However, the position may be detected by extracting an image of the treatment instrument 301 using an image obtained by turbidity correction, the position may be detected by extracting the marker sections using an IR image obtained by applying infrared light as special light of the treatment instrument, or the position may be detected by extracting the marker sections using a learning model generated by machine learning such as deep learning.

Here, as for the turbidity correction, the haze correction method described in Japanese Patent No. 6720012 or Japanese Patent No. 6559229 can be applied by replacing the haze with turbidity. Specifically, a local histogram is generated by estimating a turbid component. Thereafter, the turbidity generated region is corrected by calculating a correction coefficient based on the histogram and correcting the contrast.

In the third embodiment, the example in which the treatment by the user is assisted by image display has been described, but a configuration in which sound or light is output to assist the treatment may be adopted. In the case of assisting the treatment using sound or light, for example, the output is changed according to a distance between the treatment instrument 301 and the treatment target position. Specifically, the shorter the distance between the treatment instrument 301 and the treatment target position, the greater the sound (light intensity). Further, in a case where it is determined that the treatment instrument position coincides with the treatment target position and the treatment instrument 301 has reached the target position, output automatic stop for stopping the output may be executed.

### Other Embodiments

Various inventions can be made by appropriately combining the plurality of constituent elements disclosed in the first to third embodiments described above. For example, some constituent elements may be deleted from all the constituent elements described in the first to third embodiments.

In addition, in the support data display according to the first to third embodiments, the display form of the support data, for example, display/non-display of the support data, emphasis/reduction in the support data display, and the like may be switched according to the degree of turbidity detected by the turbidity detector 223 so that the surgeon can easily grasp information.

Further, in the first to third embodiments, the configuration in which the control unit that controls each device such as the endoscope 201 and the treatment instrument 301 is individually provided as the control device has been described, but a configuration in which one control unit (control device) collectively controls each device may be adopted.

In the first to third embodiments, the example in which white bone powder generated by pulverizing bone causes cloudiness has been described, but the disclosure can be applied to treatment or the like in which white particles cause cloudiness in addition to the bone powder.

In addition, the above-described “unit”, and “circuit” in the first to third embodiments can be read as “means”, “circuitry”, “section”, or the like. For example, the control unit can be read as a control means or a control circuit.

In addition, the program to be executed by each device according to the first to third embodiments is provided by being recorded as file data in an installable format or an executable format in a computer-readable recording medium such as a CD-ROM, a flexible disk (FD), a CD-R, a digital versatile disk (DVD), a USB medium, or a flash memory.

The program to be executed by each device according to the first to third embodiments may be stored in a computer connected to a network such as the Internet and provided by being downloaded via the network. Further, the program to be executed by an information processing apparatus according to the first to third embodiments may be provided or distributed via a network such as the Internet.

Further, in the first to third embodiments, signals are transmitted and received by wireless communication. However, for example, wireless communication is not necessary. Signals are transmitted from various devices via a transmission cable. However, wired communication may be used.

Note that, in the description of the flowcharts in the present specification, the order of processing necessary for carrying out the disclosure is not uniquely determined by the expression illustrated in the flowcharts. That is, the order of processing in the flowcharts described in the present specification can be changed to the extent that it is consistent.

Although some of the embodiments of the present application have been described in detail with reference to the drawings, these are merely examples, and the disclosure can be implemented in other forms subjected to various modifications and improvements based on the knowledge of those skilled in the art, including the aspects described in the disclosure.

As described above, the treatment system and the method of operating the treatment system according to the disclosure are useful for suppressing the influence on the surgery caused by the turbidity in the perfusate.

According to the disclosure, it is possible to reduce the influence on surgery due to turbidity in a perfusate.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment system comprising:
a treatment instrument configured to cut living tissue in a liquid;
an endoscope configured to capture an endoscopic image that includes an image of the treatment instrument and an image of the living tissue;
a support data storage configured to store, as support data to assist in a cutting treatment, at least one of:
data relating to a posture of the treatment instrument and image data on a location of a treatment section of the treatment instrument;
a support data generator configured to generate support data to be displayed on a display based on the support data stored;
a turbidity detector configured to detect a degree of cloudiness of the liquid; and
a controller configured to:
control the display to display the support data together with the endoscopic image; and
switch a display form of the support data generated by the support data generator based on the degree of cloudiness of the liquid detected by the turbidity detector.

2. The treatment system according to claim 1, wherein
the support data storage is configured to temporarily store data regarding a current position of the treatment instrument, and
the support data generator is configured to generate, based on the support data, display data as support data, the display data being based on:
each piece of positional information regarding the current position and a cutting completion position of the treatment instrument, and
a depth cut by the treatment instrument.

3. The treatment system according to claim 1, further comprising:
an image processing unit configured to extract image data of at least a part of the treatment instrument from the image captured to perform enhancement processing, wherein:
the support data storage is configured to temporarily store the image data extracted for the enhancement processing, and
the support data generator is configured to generate, as support data, display data based on the image data extracted for the enhancement processing.

4. The treatment system according to claim 1, wherein
the treatment instrument includes a marker section that is provided on a side where the living tissue is cut and emits scattered light, and
information indicating a position of the treatment instrument with respect to the living tissue is obtained using a reflected image of light in the marker section.

5. The treatment system according to claim 4, wherein
a process of emitting scattered light is retroreflection processing.

6. The treatment system according to claim 4, comprising
an image processing unit configured to extract image data of the marker section from an image captured by the endoscope to generate an enhanced image of the marker section based on the image data extracted for enhancement processing, wherein
the controller is configured to control the display to display the enhanced image.

7. The treatment system according to claim 1, wherein
the support data generator generates a guide image including an image captured by the endoscope, an entire circumferential image obtained by capturing an entire circumference of the living tissue, and the support data.

8. The treatment system according to claim 1, wherein
the treatment instrument is an ultrasound treatment instrument.

9. The treatment system according to claim 1, wherein
when the treatment instrument cuts the living tissue, turbidity occurs in the liquid, and
the turbidity in the liquid is caused by bone powders generated when a bone is cut with ultrasound.

10. The treatment system according to claim 1, wherein
when the treatment instrument cuts the living tissue, turbidity occurs in the liquid, and
the turbidity in the liquid is due to white particles generated from cutting the living tissue.

11. A method of operating a treatment system, the treatment system including:
a treatment instrument configured to cut living tissue in a liquid;
an endoscope configured to capture an endoscopic image that includes an image of the treatment instrument and an image of the living tissue;
a support data storage configured to store, as support data to assist in a cutting treatment, at least one: of data relating to a posture of the treatment instrument and image data on a location of a treatment section of the treatment instrument;
a support data generator configured to generate support data to be displayed on a display based on the support data stored;
a controller; and
a turbidity detector configured to detect a degree of cloudiness of the liquid, the method comprising:
displaying, with the controller, the support data together with the endoscopic image; and
switching, with the controller, a display form of the support data generated by the support data generator based on a degree of cloudiness of the liquid detected by the turbidity detector.
